(19) United States
(12) Patent Application Publication
(10) Pub. No.: US 2015/0196369 A1
(43) Pub. Date: Jul. 16, 2015

(54) SYSTEM, METHOD AND DEVICE EMPLOYING FIDUCIALS FOR MEDICAL INTERVENTION

(71) Applicant: Neil GLOSSOP, Toronto (CA)

(72) Inventor: Neil GLOSSOP, Toronto (CA)

(21) Appl. No.: 14/596,937

(22) Filed: Jan. 14, 2015

Related U.S. Application Data
(60) Provisional application No. 61/927,278, filed on Jan. 14, 2014.

(51) Int. Cl. A61B 19/00; A61B 18/04; A61B 18/20; A61N 5/10; A61B 5/00; G01R 33/28; A61B 6/02; A61B 6/03; A61B 6/00; A61B 5/05; A61B 8/08; A61B 8/12; A61B 5/11; A61B 10/04; A61M 25/01; A61F 2/30; A61F 2/24; A61B 5/06; A61F 2/00; A61B 18/02

(52) U.S. Cl. CPC A61B 19/54; A61B 19/5244; A61B 18/02; A61B 18/04; A61B 18/20; A61N 5/10; A61B 5/4839; G01R 33/287; A61B 6/025; A61B 6/037; A61B 6/032; A61B 6/4085; A61B 5/0515; A61B 8/481; A61B 5/0507; A61B 8/12; A61B 5/0066; A61B 5/1112; A61B 10/04; A61M 25/0108; A61M 25/0127; A61F 2/30; A61F 2/24; A61B 5/06; A61F 2/0063; A61B 2019/5287; A61B 2019/5475; A61B 2019/5445; A61B 2019/5429; A61B 2019/5458; A61B 2019/5462; A61B 2019/5483; A61B 2019/5495; A61B 2019/5276; A61B 2019/524; A61B 2019/5236; A61B 2019/5408; A61B 2019/5416; A61B 2019/5487; A61B 2019/5491; A61B 2019/5234; A61B 2010/045

(57) ABSTRACT

A system, method and devices are described that use fiducials to help localize and treat a region of tissue. The fiducials are cooperative with at least one imaging modality, but may also be located using a second physical method to determine the proximity of a seeker probe to the fiducials. The fiducials may also contain electronics and subsystems to enable them to be used to deliver therapy to the region of tissue near the fiducial.

### FIELD OF INVENTION

Embodiments of the invention relate to methods, systems, and devices for assisting or performing guided biopsy, medical therapy and intervention using localization markers or fiducials implanted into tissue.

### BACKGROUND

Medical procedures rely heavily on imaging for guidance of procedures, especially those that are minimally invasive such as needle procedures. Needle and catheter procedures are routinely performed to deliver drugs, take tissue samples, or perform therapy. Therapies may include tissue ablation therapies such as radiofrequency ablation, cryosurgery, photodynamic therapy, brachytherapy and microwaved ablation; implant of a device such as an artificial valve, stent, tube, radioactive seed or electrode; establishment of a channel or pathway such as a shunt; or bypass or closure or surgical resection of a portion of tissue.

When performing interventional procedures, the physician must know the position of the instruments relative to the tissue of interest. While this is sometimes obvious, e.g. direct visualization of an obviously differentiated tissue type, it is often not. Sometimes the diseased tissue may not look different than the normal surrounding tissue, sometimes the instruments may not be directly visualized, sometimes the tissue may not be directly visualized or all of the above. In addition, flexible instruments (such as catheters) may easily be distorted during their intended use, rendering it almost impossible to ascertain the exact location and orientation of the device without assistance. Implanted devices such as brachytherapy seeds may also be small and widely dispersed so it may be difficult to determine the location of all the seeds.

In many cases these procedures may be carried out with the assistance of volumetric imaging such as Computed Tomography (CT), magnetic resonance imaging (MRI), Positron Emission Tomography (PET), and the like. They may also be carried out using optical imaging (e.g. through an endoscope), ultrasound imaging (US), X-ray imaging, etc.

Imaging modalities such as US or X-ray may be used during the interventional procedure to help locate the tissue and/or instruments. Although they may be more portable and convenient than volumetric modalities these imaging devices may be of lower resolution and may not offer as much information.

The current state of the art does not allow accurate resection or easy targeting of a biopsy needle into a potential cancer site seen under volumetric methods once the patient has been moved out of the scanner. While possible to perform the intervention in the scanner itself, this is time consuming, inconvenient and costly and most interventions occur in locations that include only simpler imaging devices like US or X-ray.

In other cases, such as those performed under x-ray, the anatomy may be visible during doses of contrast injection and while the x-ray beam is on. This may expose the surgical team and patient to frequent doses of ionizing radiation and the patient to high doses of nephrotoxic contrast agents.

In some cases such as those performed using ultrasound, the anatomy may be poorly visualized or presented in a form that make it difficult for the physician to interpret. Some lesions or anatomy may not be suited to ultrasound at all.

In addition, it is sometimes desirable to perform a minimally invasive procedure to minimize the chance of severe complications that sometimes accompany surgery. By precisely targeting focal cancer lesions, treatments may be possible without subjecting the patient to a large, invasive procedure that would otherwise be poorly tolerated. Only the diseased tissue may be targeted, and healthy tissue spared.

In most cases, the location of an instrument must be precisely known in order to properly treat the patient. For example, the location of a stent located on a catheter must be known before it is deployed. Similarly, the locations of a catheter delivered or transapically delivered heart valve must be known prior to deployment. The location of the ends of internal feeding tubes or PICC lines must also be understood relative to the anatomy to ensure they arrive in the intended location. Other examples include placement of biopsy needles prior to sampling, placement of needle therapy devices as listed above or devices for marking of tumor boundaries for use in later surgery or radiation therapy. When a plurality of devices are implanted such as brachytherapy seeds, it is helpful to know where each is located to ensure the therapy is correctly administered and that they are in the correct location.

In some surgical cases, imaging is used in the preparation for surgery. The imaging may be used to place a localization marker or fiducial that can then guide subsequent surgery to the appropriate location. This is commonly used with a localization wire placed prior to breast surgery. More recently radioactive localization seeds can be placed at the target tissue and then during surgery a Geiger counter may be used to guide the surgeon to the target tissue without the need for wire localization.

Accordingly there exists a need to solve these and other problems to facilitate procedures that require the localization of a therapeutic surgical instrument relative to anatomy. To assist in such cases, implanted fiducials may be used to mark anatomy of interest.

Fiducials or localization markers are normally passive devices that are visible from the surface or visible under at least one imaging modality or one localizing device (e.g., Geiger counter) that are temporarily or permanently in a patient or device. Fiducials are often placed into a pathology that is visible using an imaging modality to assist locating of the pathology when it cannot be directly seen under another modality. For example, fiducials may be placed using MRI in order to locate a tumor for surgical resection at a later time. The tumor may not be visible under direct visualization so the fiducials may be beneficial to denote the tumor.

Once in place, fiducials may denote a region wherein a subsequent procedure may be performed, assist in registration for a computer assisted image guided surgical procedure, assist in registration for radiation therapy, denote a boundary, denote a critical structure or assist in measuring tissue motion. Being in the location of the pathology, there is the potential for the fiducial to also perform therapy as in the case of brachytherapy. Fiducials may also be embedded into other instruments or devices to denote the position and/or orientation of the instrument, e.g. the tip of a needle, catheter or other device.

During treatment or biopsy, fiducials may themselves be difficult to locate, extract or identify at some later time. It can be inconvenient or time consuming to do so. If a fiducial is placed using a minimally invasive approach, there may be no visible mark as to where the fiducial is located. This means that when the tumor is to be treated or biopsied, there is almost no indication as to where the fiducial is located and it may be necessary to resort to imaging or exploration in order to locate the fiducials.

In breast surgery, implanted radioactive fiducials or transcutaneous wires are widely used to locate non-palpable lesions. These are implanted under image guidance such as MRI, CT or tomosynthesis, and then located during surgical resection of the lesion. There are multiple problems with localization using these techniques that make them inefficient and less than ideal.

Radioactive ¹²⁵I and ⁹⁹TC fiducials used as point sources can be detected with a hand held gamma probe to guide the surgeon to the fiducial and tumor site. Radioactive fiducials require proper radioactive device handling techniques and licenses, and can be logistically challenging for short half-life isotopes. Patients and physicians are uncomfortable with devices that emit ionizing radiation.

Wire fiducials also suffer from several problems. The entry point that the radiologist selects when placing the wire may be distant from the lesion. Surgical incisions must follow the wire which leads to a more invasive path to the target. Determining precisely what tissue should be removed is also an issue with wire fiducials, and surgeons frequently remove non-cancerous tissue. Patients are uncomfortable with wires extending out of breast tissue.

Accordingly there exists a need to solve these and other problems to facilitate procedures that require the localization of fiducials placed within tissue. For example, there exists a need to locate fiducials once placed into a tumor using an imaging modality so that the tumor may be extricated without having to reuse the imaging modality, which may be incompatible with the surgical setting. There exists a need for a non-radioactive fiducial that may be easily and efficiently located using a hand held device. There exists a need to perform procedures using implanted fiducials that may be activated as required in order to destroy tumors.

### SUMMARY

Embodiments described herein solve these and other problems by providing methods, systems, and devices employing fiducials. In a first aspect, a method for performing a guided interventional procedure with the aid of one or more fiducials is disclosed. A fiducial is a device configured to emit a signal in response to a signal transmitted by a probe. In one embodiment the method includes placing a fiducial, localizing the fiducial, and performing a procedure on a tissue.

In one embodiment, the fiducial placement may include performing an imaging study on a tissue, identifying a lesion in the tissue, and placing one or more fiducials in the tissue. The fiducial localization may include placing a probe configured to detect the fiducial in proximity to the one or more fiducials in the tissue, detecting the one or more fiducials, and indicating the position of the probe relative to the one or more fiducials.

In a second aspect, a fiducial device is disclosed. In one embodiment, the fiducial device is configured to emit a signal indicative of its location relative to a probe in response to a signal transmitted by a probe where the fiducial is comprised of a reflective device capable of reflecting an incident signal, a device designed to transmit a signal in response to an incident signal, or a device designed to modify a property of a signal generating system. The fiducial device further includes an element configured to constrain the fiducial within a tissue.

In a third aspect, a system for performing a guided interventional procedure is disclosed. In one embodiment, the system comprises a computer, a tracking device, an imaging device, and one or more fiducials. The one or more fiducials can be configured to emit a signal in response to a signal transmitted by a probe indicative of its location relative to the probe.

The various objects, features, and advantages of the embodiments will be apparent through the detailed description and the drawings attached hereto. It is also to be understood that the following detailed description is exemplary and not restrictive of the scope of the embodiments.

### BRIEF DESCRIPTION OF THE DRAWINGS

FIG. 1 is a schematic view of a system for assisting or performing image-guided procedures that uses implantable fiducials;

FIG. 2 is a view and close-up of an implantable fiducial for assisting in image guided procedures that uses RFID technology;

FIG. 3 is a view and close-up of an implantable fiducial for assisting in image guided procedures that uses reflective technology;

FIG. 4 is a view of a probe used to located a fiducial;

FIG. 5 is a view of a probe with separated transmitter for locating a fiducial;

FIG. 6 is a flowchart for placing a fiducial and locating it during a surgical procedure;

FIG. 7 is a view of an energy emitting fiducial;

FIG. 8 is a view showing a plurality of energy emitting fiducials;

FIG. 9 is a view of a fiducial with identification features present;

FIG. 10 is a view of two energy emitting fiducials with overlapping treatment zones; and

FIG. 11 is a flowchart for placing and activating therapy fiducials.

### DETAILED DESCRIPTION

Embodiments as described herein provide devices, systems, and methods for assisting or performing guided interventional procedures using fiducials to mark or denote regions and/or to provide therapy to the region. Guided interventional procedures may include surgical resections, biopsies or focal ablation of a tumor or suspected tumor, placement of a feeding tube, marking of a tumor extent, marking of an aspect of anatomy that is either a target or a critical location that must be avoided. Guided interventional procedures may also include marking and manipulation of certain bones or fragments thereof. It may include marking or identifying instruments such as the tips of needles, brachytherapy seeds, fiducial markers, feeding tubes, catheters including device delivery catheters and drug delivery catheters. It may include marking of devices such as stents, stent grafts, sponges or other such devices.

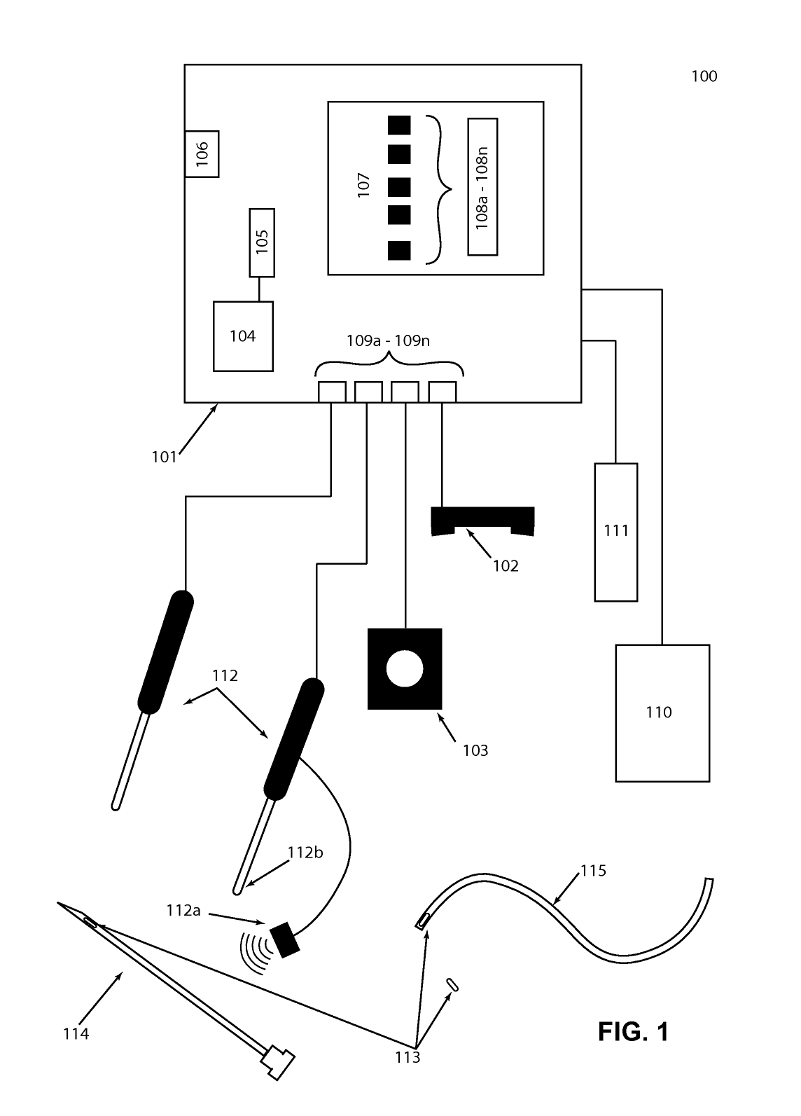

FIG. 1 illustrates a system 100 for assisting or performing procedures. System 100 may include a computer element 101, a tracking device 102, an imaging device 103, a needle assembly 114, or other elements.

Computer element 101 may include a processor 104, a memory device 105, a power source 106, a control application 107, one or more software modules 108a-108n, one or more inputs/outputs 109a-109n, a display device 110, a user input device 111, and other elements. In some embodiments, the processor 104 may be configured to perform the features and functions of the invention as described herein. Memory device 105 or other memory or data storage elements or methods may store data or otherwise provide instructions to the processor 104.

Computer element 101 may be or include one or more servers, personal computers, laptop computers, mobile computers, tablet computers, or other computer devices. Computer element 101 may receive, send, store, or manipulate data necessary to perform any of the processes, calculations, image formatting, image display, or other operations described herein. Computer element 101 may also perform any processes, calculations, or operations necessary for the function of the devices, elements, instruments, or apparatus described herein.

In some embodiments, computer element 101 may host a control application 107. Control application 107 may comprise a computer application which enables one or more software modules 108a-108n. Software modules 108a-108n may enable processor 104 to receive (e.g., via a data reception module), send, or manipulate data regarding the anatomy of a patient, one or more objects, or other data. This data may be stored in memory device 105 or other data storage location. In some embodiments, software modules 108a-108n enable processor 104 to receive live or stored data (e.g., via the data reception module), send, or manipulate data regarding the location, position, orientation, or coordinate of a position indicating element (e.g., sensor coils or other position indicating elements) received by position sensor 102. This data may be stored in memory device 105 or other data storage location.

In some embodiments, software modules 108a-108n such as, for example, a display module, enable processor 104 to produce, format, or reformat one or more images, position/orientation/location data, or other data. Images may be displayed on a display device 110. In some embodiments, processor 104 displays one or more live images. In some embodiments, display device 110 may display audio data in addition to or instead of visual data. Such an audio display may produce tones or other indicators regarding the system.

Software modules 108a-108n such as, for example, the display module, may enable the generation and display of images of the anatomy of the patient with the position or orientation of an instrument, fiducials or both superimposed thereon in real time (such that motion of the tracked instrument within the anatomy of the patient is indicated on the superimposed images) for use in an image-guided procedure. In some embodiments, software modules 108a-108n such as, for example a display module, enables processor 104 to produce markings, lines, circles, spheres, letters, numbers or other indicators on an image of the anatomy of a patient. These markings may indicate features such as the boundaries of another image stored in memory device 105. In some embodiments, software modules 108a-108n such as, for example, a mapping module, enables processor 104 to map a target lesion (e.g., a cancerous region) or other portion of a patient's anatomy or to perform other operations related to a map of the target lesion or portion of the patient's anatomy. In some embodiments, software modules 108a-108n such as, for example, display module generates and display (e.g., on display device 110) the position of a puncture needle relative to a location in the target lesion, a projected path of the puncture needle including a path the puncture needle will follow if the puncture needle is extended past a distal end portion of the needle guide, a point at which the puncture needle will intersect the target lesion if the projected path of the puncture needle intersects the determined path of the target lesion, and an indicator of the closest approach from the puncture needle to the target lesion if the projected path of the puncture needle does not intersect tissue not intended to be treated or biopsied. Likewise it may indicate the proximity of a probe or needle to another device such as a fiducial or other needle or device.

Display device 110 may include a computer monitor or other visual display device such as, for example, an LCD display, plasma screen display, cathode ray tube display, or other display device. Input device 111 may include a mouse, a stylus, a keyboard, a touchscreen interface (which may be associated with or integrated with display device 110), a voice activated input device (including a microphone and associated voice processing software), or other device wherein a user (e.g., a physician performing a procedure or assistant thereto) can provide input to system 100 or its components.

In some embodiments, tracking device 102 may be used. It need not be directly operatively connected to computer element 101, but data may be sent and received between tracking device 102 and computer element 101. Probe 112 may be operatively connected to computer element 101 via an input/output 109a-109n. Probe 112 may include an electromagnetic tracking device, micropower impulse radar (MIR) or other radar device, optical range finder, a metal detector, an ultrasonic device, or other type of measurement device capable of determining the spatial distance between a fiducial and the probe. Probe 112 may incorporate a radiofrequency identification (RFID) device. Probe 112 may be used to obtain data regarding the three-dimensional location, position, coordinates, or other information regarding one or more fiducial elements 113 within or around an anatomical region of the patient. Probe device 112 may provide this data or information to computer element 101.

Imaging device 103 may include x-ray equipment, computerized tomography equipment, positron emission tomography equipment, magnetic resonance imaging equipment, fluoroscopy equipment, ultrasound equipment, an isocentric fluoroscopic device, a rotational fluoroscopic reconstruction system, a multislice computerized tomography device, an intravascular ultrasound imager, an optical coherence tomography (OCT) device, an optical imaging device, a single photon emission computed tomography device, a magnetic particle imaging device, or other imaging/scanning equipment.

Fiducial element 113 may include one or more of a retroreflective device such as a “radar corner retroreflector,” optical corner reflector, a “cats eye retroreflector,” phase conjugate mirror, etc. Alternatively, fiducial element 113 may include simpler diffusely reflective surfaces that may produce a spatially broader reflective signal.

In an embodiment, metal detection technology may be employed so that fiducial element 113, which may be constructed from a metallic substance, can be detectable using one or more of the commonly used metal detection technologies that may include Very Low Frequency (VLF), Pulse Induction (PI) and/or Beat-frequency Oscillation (BFO). In this embodiment, a signal may be received by the receiver that indicates the distance and/or direction between the detector antenna or emitter and the fiducial. In an embodiment the fiducial element itself may not necessarily be made entirely of metal but may contain metal elements including coils and antennas within a metal or nonmetal housing. Fiducial element 113 may also contain elements such as a coil, inductive-capacitive (LC) or resitive-inductive-capacitive (RLC) circuit that may be tuned to resonate at a specific frequency that may be generated by the detector or is otherwise detectable by the detector. When using a plurality of fiducials, each may be tuned to a different frequency by appropriate modification of the R, L or C component of the circuit enabling the system to differentiate between them. Differentiation may also be achieved through appropriate design of the geometry of the fiducial. This may also be achieved by manufacturing fiducials from different materials to assist in differentiating between them. In these cases, the conductivity or other properties may be slightly modified through the addition or change in the proportion of constituent elements. Fiducials may be also made to contain different amounts of conductive material housed within a commonly sized carrier.

Fiducials may be individualized by altering the size, shape, surface area, material, electrical, magnetic, thermal, optical, sonic, chemical, and other properties of all or part of each fiducial that must be differentiated from another fiducial. Non-limiting examples of properties of fiducials that may be altered (either alone or in combination with one another) in order to assist in differentiating individual or groups of fiducials from one another include changes to the fiducial's electrical conductivity, electrical impedance, electrical capacitance, dielectric permittivity, magnetic inductance, magnetic susceptibility, magnetic permeability, diamagnetism, paramagnetic properties, gyromagnetic properties, ferromagnetic properties, thermal conductivity, heat capacity, reflectivity, absorptivity, transmissivity, polarization, spectral absorptivity, fluorescence, refractivity, or other detectable property of the fiducial.

Fiducial element 113 may also be constructed as an RFID tag, in which an electromagnetic field is used to power the tag by electromagnetic induction. The tag may then act as a passive transponder to emit microwaves, UHF radio waves or other signals. Fiducial element 113 may also include a local power source such as a battery to power it and allow it to send and receive signals.

In some embodiments, fiducial elements 113 may be placed on or integrated in a needle 114, biopsy needles (not shown), catheter 115, clamp (not show), heart valve (not shown), total joint arthroplasty (not shown), intramedullary nail (not shown), medical probe (not shown), mesh (not shown), or other medical instrument or device meant for permanent or temporary implantation. Probe 112 may include integrated emitting and receiving components or as shown in 112, these functions may be separated with emitter 112a separated from the receiving aspects of the probe 112b.

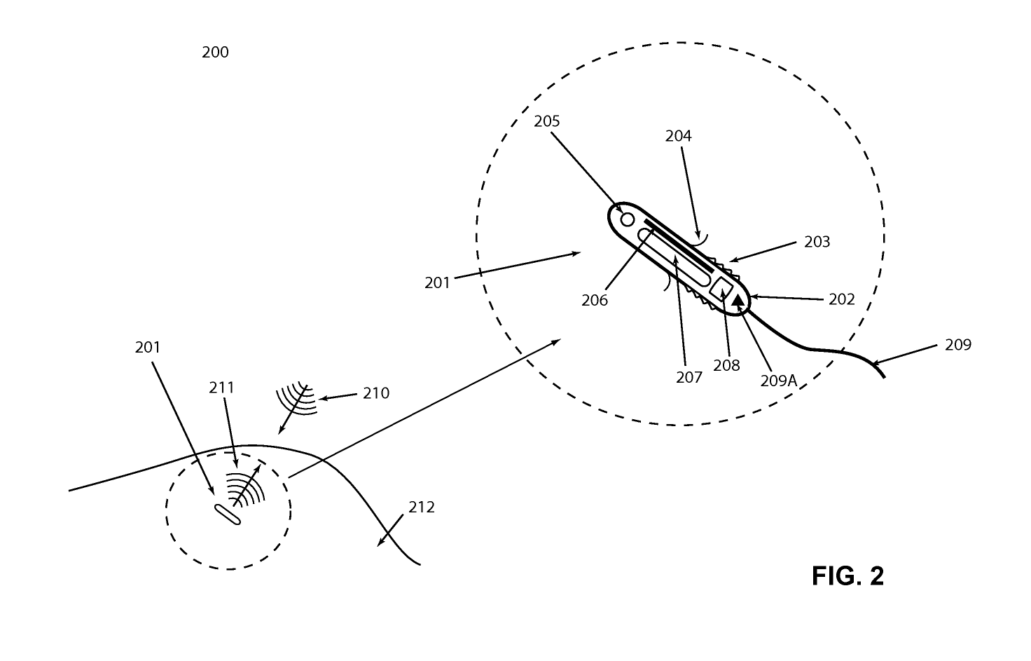

Turning now to FIG. 2, an embodiment including an embedded fiducial is shown as 200. Fiducial 201 is shown embedded within a tissue aspect 212 which may include for example breast tissue, liver tissue, pancreas, prostate, brain, bone, or any other organ or tissue. The fiducial may be placed within the organ surgically, or by using a delivery system such as a needle (e.g., cannula), so that it remains within the organ.

The fiducial 201, shown enlarged in the inset, may be configured with an outer case or housing 202 which may be constructed of a biocompatible and structurally stable material such as glass, stainless steel, titanium, plastics such as polyethylene, polyetheretherketone (PEEK), ethylene vinyl acetate, polyphenylsulfone (PPSU), polysulfone (PSU), a permeable material that may permit a pharmaceutical or other agent to permeate therethrough, (or only a portion is permeable or rendered permeable upon activation) etc. In an embodiment, the fiducial may be MRI compatible or MR safe. In an embodiment, the fiducial may be radio-opaque or radiolucent. In an embodiment, the fiducial 201 may include elements such as ridges, 203 which may serve to help constrain the fiducial 201 within the tissue 202. In an embodiment, the fiducial may include one or more deployable barbs 204 that may serve to stabilize the fiducial 201 in the tissue 202. In an embodiment, the fiducial 201 may include a battery 205 that may power or activate electronic components 208 encapsulated within the fiducial. In an embodiment, the fiducial 201 may include an antenna 206 which may be used to assist in converting electric power from the fiducial into a signal such as radio waves. In an embodiment, the fiducial 201 may include a second antenna 207 that may receive power projected by an external device in order to supply electrical energy for electrical components 208 encapsulated within the fiducial 201. In an embodiment the receive antenna 206 and 207 may be the same antenna. In an embodiment, fiducial 201 may incorporate a tether 209 made of a wire or fibrous material to facilitate easy removal if desired, or to connect it to other fiducials. In an embodiment, the fiducial may serve as a carrier for one or more quantities of a metallic material 209A.

In an embodiment, fiducial 201 may be delivered into tissue 202 using a hollow needle or cannula. The cannula may then be inserted into the tissue, a plunger depressed, and the fiducial(s) expelled into the tissue.

In an embodiment, the fiducial 201 may be powered by a power pulse 210 that is emitted by a power emitter. In an embodiment the fiducial 201 may emit a signal 211 (coded or un-coded) regarding some fixed information stored within the fiducial, for example a serial number, or some information regarding the environment of the fiducial for example its temperature, location, externally delivered energy such as radiation, or a combination of this information.

In an embodiment, a plurality of fiducials 201 may be used, each emitting a unique coded signal 211. Each fiducial can therefore be uniquely identified by this coded signal.

In an embodiment, the fiducial 201 may be configured to re-broadcast the incoming signal 210 in either the identical form as the incident signal 210 or as a variation of the incident signal, as signal 211. In an embodiment, signal 211 may be augmented by additional information that carries identification or measured attributes of the fiducial. In an embodiment, fiducial 201 may be configured to continually emit a signal 211 that can be picked up by a suitable receiver.

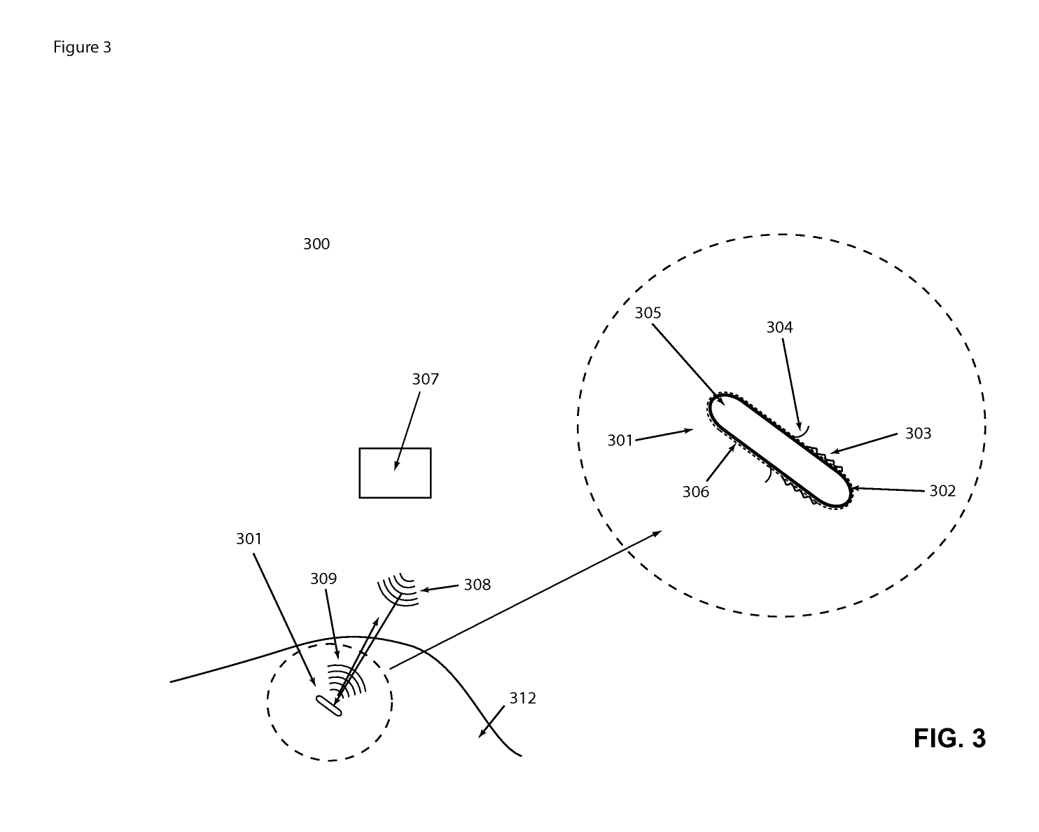

Turning now to FIG. 3, which illustrates a fiducial 301 (e.g., a retro-reflective fiducial 301) embedded in tissue 312. In an embodiment, like the previous figure, the fiducial may include stabilization elements 303 and 304 representing the ridges and deployable barbs of the previous figure. Reflective fiducial 301 may be made of materials 302 similar to fiducial 201 of FIG. 2, i.e. stainless steel, biocompatible plastics, titanium and the like. Fiducial 301 may be coated with a material 306 to render it reflective under modalities that may include ultrasound, radar, laser light or other electromagnetic radiation. In an embodiment, the material may include a glass bead coating, a prismatic coating, an Echo-Coat™ coating or some other coating that will render the fiducial reflective or retro-reflective of incoming energy 308 so that it reflects off the coating 306 of fiducial 301 or off of fiducial 301 itself. Upon application of energy 308, it is preferentially reflected from coating 306 so that the fiducial appears to be a source of the reflected energy 309. Using an appropriate receiver 307 the relative proximity, the location and/or orientation of the fiducial may be determined.

In a similar manner, additional technologies may be used as an alternative to radar and RFID. In such cases the technology is adjusted appropriately. For example a purely optical system may use a fiducial similar to 301 but coated with a coating 306 that will reflect the appropriate wavelength of incident energy 308. In an embodiment, the coating 306 may include a fluorescent material that emits light at a frequency other than that illuminating the fiducial. Other materials may include those that make use of other nonlinear optical properties such as Raman scattering or two-photon processes.

In an embodiment, the fiducial may itself be configured to emit energy rather than reflect it. Fiducial 301 may include a light source, a radio source, a microwave source etc.

A fiducial based on sound may also be used. Such a fiducial may emit an audible click, chirp, or continuous tone that can be used to help localize it with an externally held microphone receiver.

A fiducial that disturbs an electromagnetic filed may also be used. In this case an electromagnetic field may be created and the presence of the fiducial may be revealed by measuring the alteration in this field.

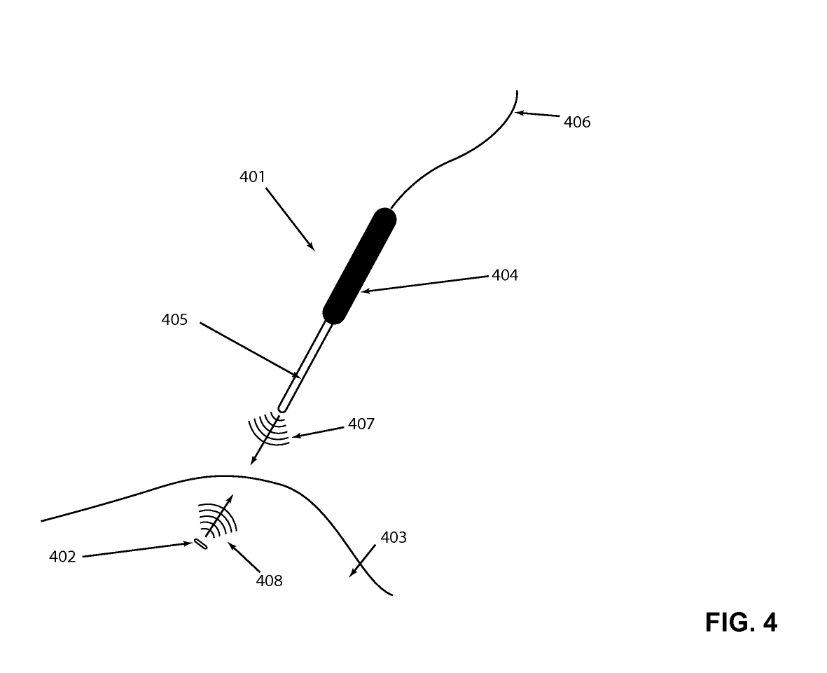

FIG. 4 shows a possible arrangement of an embedded fiducial with a “seeker” probe. Here a probe 401 is shown in proximity to a fiducial 402 that has been embedded in tissue 403. Probe 401 consists of a handle 404, a stem 405 and an optional cable 406 that leads back to the control unit. In an embodiment, cable 406 may be replaced by a wireless transmission system. In some configurations, the seeker probe 401 or parts thereof may be placed internally in the patient or tissue. In an embodiment, a catheter, needle, or internal probe may act as the probe 401.

Stem 405 may contain one or more antennae, light sources, speakers or other components. In an embodiment, the handle 404, stem 405 or other part of the probe may contain electronic components such as one or more antennae, speakers, light sources, indicators, switches, buffer, amplifier analog to digital converter etc.

Using one of the aforementioned technologies, the probe may produce a signal 407 that may include a power or energy signal which may be received by the components in the fiducial and cause a second signal 408 to be emitted, re-emitted, disturbed-by or reflected from the fiducial. The signal 408 may be picked up by other receiving (or receiver) components within the probe 401 which may include antennas, microphones, light sensitive devices (such as charge coupled devices or photodiodes), magnetometers or other magnetically sensitive component, eddy current detectors, capacitive sensor, capacitive displacement sensors, inductive sensors, etc.

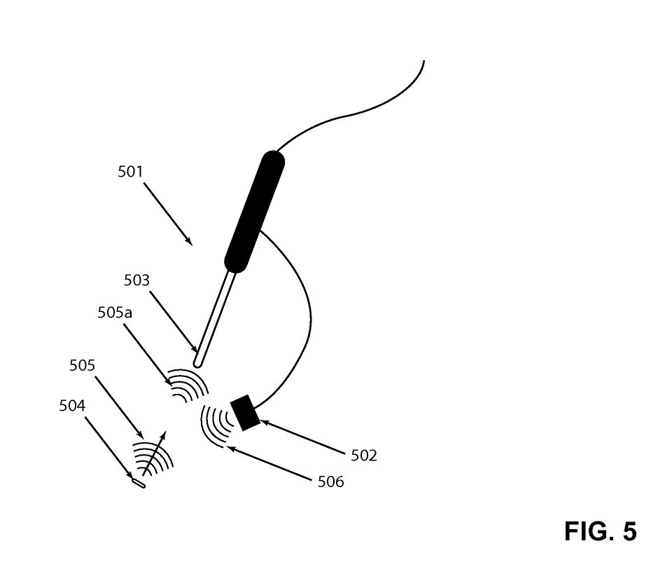

In an embodiment shown in FIG. 5, the probe is indicated as 501. The transmission component 502 may be separated from receiver component 503 of the probe. For example, a power generator or ambient field generator 502 may be placed in the room that broadcasts an energy field 506 such as an EM field. The fiducial 504 may cause a signal 505 to be emitted, disturbed or reflected until it impinges upon the receiver elements within the probe as shown by signal item 505a impinging upon receiver element 503. In an embodiment, the seeker probe 501 or parts thereof may be configured as a catheter, needle or other type of internal probe.

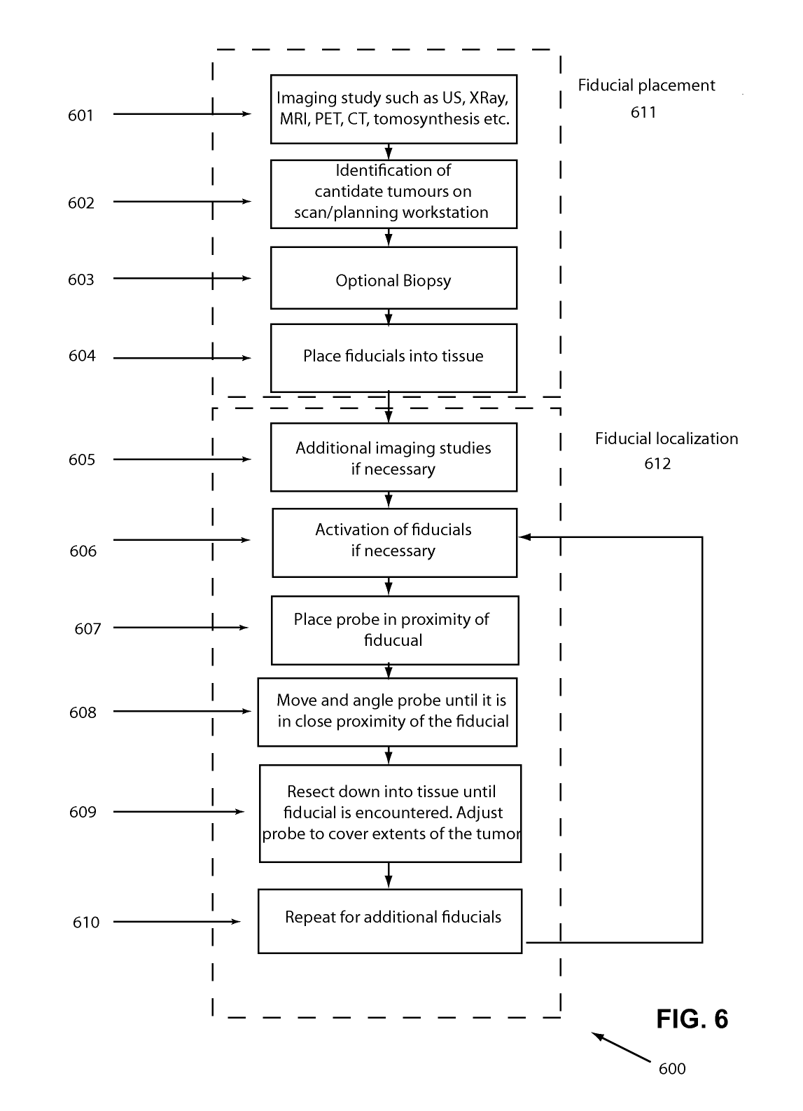

In FIG. 6, flowchart 600 illustrates a workflow for a method of using embodiments of the invention. The workflow is divided into fiducial placement 611 and fiducial localization 612. In the fiducial placement procedures portion of the workflow, one or more imaging studies 601 may first be performed. Such imaging study may consist of an ultrasound, contrast enhanced US (CEUS), x-ray, biplane x-ray, cone beam x-ray, MRI, CT, tomosynthesis, PET, magnetic particle or other imaging study in which candidate lesions in the breast or other tissue is visible. These may include studies using magnetic resonance imaging, computed tomography, cone beam CT, positron emission tomography, ultrasound, ultrasound elastography, or magnetic particle imaging.

Next, the candidate lesions can be identified on the images as shown in item 602. The location may be identified in 3D space and may correspond to the center of each lesion, or may outline the periphery or extents of the lesion or candidate lesion.

A biopsy optionally may be performed at 603 using current techniques such as stereotactic systems including systems sold by Hologic and other companies. Such a biopsy may be assisted by an imaging device such as an x-ray system including a tomosynthesis system, CT, x-ray, ultrasound etc.

The fiducials may be placed into the lesions or around them at 604. The fiducials may be left in position until a decision is made to escalate the treatment of the patient to include surgical resection or other procedure.

If a decision were made to escalate the treatment that may include surgical resection of the site for example a breast lumpectomy, additional imaging studies 605 may be indicated which may assess the location of the fiducials relative to the lesions or candidate lesions.

In an embodiment, the fiducials may be activated if necessary. This may involve turning on the transmission component 502 from FIG. 5, for example. This is shown in the flowchart as item 606 if necessary. “Activation” in this sense is a generic term and may or may not take the form of any change in the state of fiducial itself but may equally mean activation of the system.

The probe receiver elements or transmit/receiver components may then be placed in proximity of the fiducials as shown in 607. The probe can be moved and the fiducial proximity signal determined. An indication of the probe proximity may be obtained by the electronics connected to the probe.

The probe can then be moved and oriented as indicated in 608 to a position where the electronics and display indicate the position of the probe relative to the fiducial. The user continues to move the probe until he determines that the probe is in the correct position relative to the fiducial.

Once in the correct location and orientation, a physician (or other personnel) may resect the tissue down until he encounters the fiducial as indicated in 609. The tissue margins surrounding the fiducial may also be extracted depending on the outcome of the prior imaging studies. If multiple fiducials are placed that surround the lesion, then the resection may be carried out with the assistance of the probe so that the entire region is removed, including the fiducials. Additional operations may be performed once the fiducials are located, which may include injection of drugs or other therapeutic agents; ablation using heat, laser or cryotherapy etc., placement of other fiducials, positioning of a therapeutic energy or radiation beam etc. The process then can be repeated for any additional fiducials that may be present, as indicated in 610.

In an embodiment, the fiducials may be constructed to convert the externally applied power field into another form of energy that may include heat, light, vibration, or sonic energy or any other type of energy. In this mode, the externally applied field may cause the fiducial to heat up to a temperature that will cause loco-regional heating of the tissue in which it is embedded. In another embodiment, the externally applied field may cause the fiducial to emit light so that the local environment is illuminated either uniformly or directionally. Such emission of light may be useful, for example, to activate a substance or device in the tissue (e.g., a photosensitizer or light activated drug), that can provide a desired effect when activated. In an embodiment, the externally applied field may cause the fiducial to emit sound waves or to vibrate at some frequency. In an embodiment, the externally applied field may also cause the fiducial to convert the externally applied field into another form of energy.

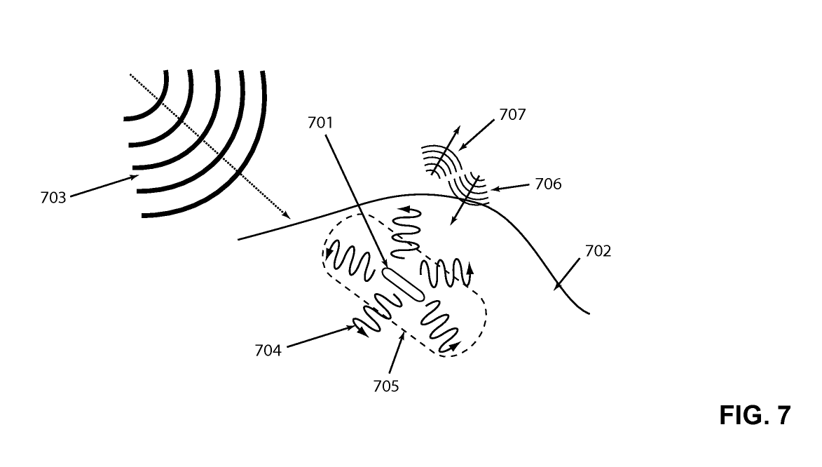

An additional embodiment is illustrated in FIG. 7, where fiducial 701 is embedded in tissue 702. An externally applied power field 703 may be applied to the region where fiducial 701 is embedded. Power field 703 can power electronics and other functions of fiducial 701. In an embodiment, power field 703 may be an ambient alternating current electromagnetic field of a prescribed magnitude and intensity. In an embodiment, power field 703 may be a gradient or other field generated by a magnetic resonance imaging system. Fiducial 701 may contain electronics that are capable of converting power field 703 into another energy form, for example heat, light, vibration, or sound energy indicated as 704. This energy permeates the local region around fiducial 701 causing for example a local increase in temperature for a heat emitting fiducial (HEF) so that after some time, the entire region around the fiducial indicated as the dashed zone 705 is elevated in temperature, possibly enough to kill the cells in this region. It is understood that although represented as a two dimensional region, in this illustration, zone 705 may be, in fact, a three dimensional shape such as an ellipsoid or other complex three dimensional shape. Zone 705 may also be permeated with light in the case of a light emitting fiducial (LEF). A sound or vibration emitting fiducial (VEF) may emit a vibration field in zone 705 around the fiducial. Other forms of energy may also be emitted by fiducial 701. In an embodiment, fiducials may emit more than one form of energy. The energy may act alone or together with other agents to alter or kill the surrounding cells. In an embodiment, fiducials that emit or transform energy may also be used to help localize the fiducials, without performing the added function of killing the surrounding cells.

In an embodiment, fiducial 701 may contain one or more pharmaceuticals, medicaments, or other substances useful in treating the tissue 702 in which the fiducial 701 is embedded. In an embodiment, fiducial 701 can be comprised of an outer case or housing (202 in FIG. 2) that is configured to permeate the substance to permeate or diffuse therethrough, or outer case or housing may partially be comprised of a material that, upon heating or vibration or other energy activation, may cause that material to become permeable or permit the substance to diffuse therethrough. This embodiment permits the precise application of a substance to a tissue such that only the area denoted by zone 705 is treated with the substance, and whereby only certain fiducials may be activated at certain times, depending on their location in the tissue 702.

In an embodiment, a command signal such as a digital signal 706 may be emitted from an externally located antenna or other device (not shown) using characteristics that are different from power field 703. In an embodiment, this signal may be used to command the fiducial to emit some kind of identification signal, to “turn on” or to perform some other action. In an embodiment, command signal 706 may be coded to a particular fiducial so that if fiducial 701 receives command signal 706 and fiducial 701 is coded to accept this particular command signal, then fiducial 706 may “activate,” “deactivate,” “change state,” or provide some kind of response signal. If the command signal does not match the signal necessary to address fiducial 701 then fiducial 701 may ignore the signal. In an embodiment, command signal may be used to turn on, turn off or adjust the energy conversion feature of the fiducial if available, or it may be used to command the fiducial to respond with some information regarding the environment of fiducial 701, for example the temperature of the fiducial or its environment. In this embodiment, the information may be sent with a response signal 707 which may include encoded information in for example a digital format. In an embodiment, the response signal may include the markers identity along with additional information. In an embodiment, externally applied field 703 may be used to encode the command signal 706 using for example by modulating externally applied field 703 as a carrier.

In an embodiment, power field 703 may be tuned to a frequency particular to a fiducial, so that it causes for example a resonance to occur within the electronics of a fiducial. In such cases the resonance circuit in the fiducial may cause it to heat up.

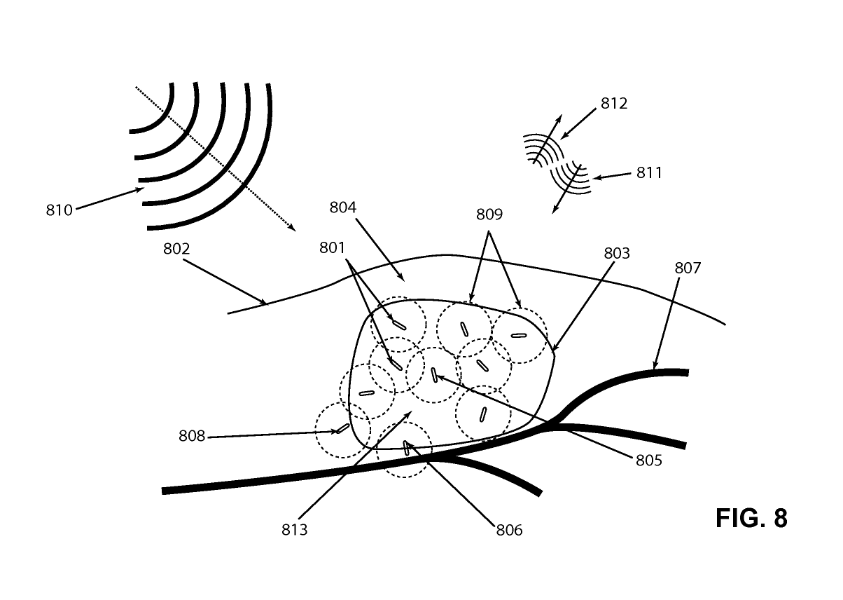

FIG. 8 illustrates an embodiment in which a plurality of fiducials 801 have been embedded into tissue 802 with the aim of treating a region of abnormal tissue for example a tumor, illustrated here as 803. Tumor 803 is embedded in a region of normal tissue 804. In treating the tumor using the embodiments described herein, the plurality of fiducials 801 can be inserted into a region in and about the tumor using needles or other minimally invasive delivery means. Because it is difficult to place the fiducials precisely, some of these will be within the tumor e.g. fiducial 805. Some fiducials such as fiducial 806 may be placed near critical structures such as blood vessels or nerves 807, or bordering tumor 803. Still others such as fiducial 808 may be entirely outside the tumor.

To safely and effectively treat tumour 803, a method such as thermal ablation may be used. In thermal ablation, the tissue temperature is raised by a heating method to a temperature that causes the cells to die. In another embodiment, a method such as photodynamic therapy may be used in which a systemic or local photosensitizer is given to the patient. When illuminated with the correct wavelength(s) of light, the agent is activated and causes cell death in the region that was illuminated.

In these cases, the regions the fiducials 801 are able to affect are generally fairly small, denoted by circles 809. In reality, the shape would be a three dimensional shape such as a sphere, ellipsoid or some other three dimensional shape. In an embodiment, the regions within the “kill zone” of these shapes 809 represents the region where tumour cells may be killed by activating the fiducial(s). Regions such as 813 may not lie within a kill zone and may be untreated if the coverage of fiducials is not sufficient. In such cases the physician may elect to introduce additional fiducials into region 813 or treat this area in some other manner.

Using the devices and methods described above, in some embodiments it is possible and desirable to address individual fiducials using command signal 811. In this way, only those fiducials in the tumor region such as 805 may be activated while those such as 808 or 806 may be deactivated. Application of the externally applied field shown as 810, would cause only the selected fiducials to undergo heating, protecting healthy tissue and critical structures while causing death of tumor cells. In an embodiment, in order to monitor the process, information signals such as 812 may be sent from the fiducial to indicate temperature, light intensity of the surrounding tissue, radiation levels or some other measure regarding the environment or status of the fiducial. In an embodiment, when the temperature or other measured aspect of the fiducial exceeds a threshold, the fiducial or adjacent fiducials or an external energy source may be turned off so that they no longer cause tissue damage. In this way, the pattern, intensity, and duration of the heating can be controlled to provide optimal destruction of the tumor 803, while preserving normal tissue 804 and critical structures 807.

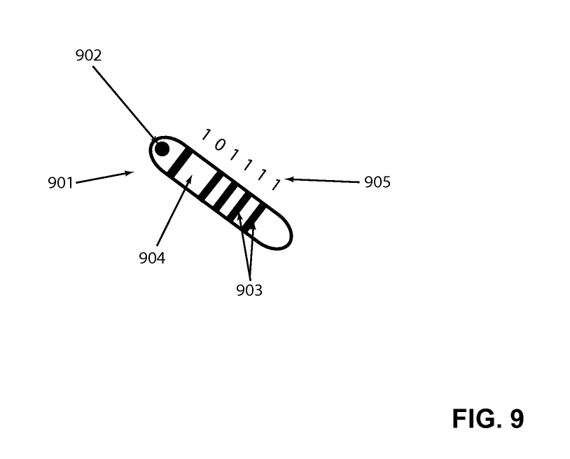

In an embodiment, shown in FIG. 9, the fiducials may include internal or external markings that are visible on x-ray or CT scan etc. that enable unique identification of each marker on the images. For example, a marker may have distinguishing features on it that it is identifiable as marker “1” when observed in images. A marker encoded electronically as a particular marker may have identification marks on it indicating that it is that marker. By enabling a one-to-one correspondence between the images of the fiducials and their internal coding, it is possible to determine which markers are in the tumor and which are outside it. The tumor may be optimally destroyed without affecting intact tissue by turning on only those fiducials within the tumor.

In an embodiment applicable to CT and X-ray, a series of radio-opaque bands may be used to identify the marker. Fiducial 901 may be equipped with a radio-opaque indicator 902 (which may be in the form of a ball, a cube, a thin layer or other indicator) to indicate the direction the code in which the code should be read. In an embodiment, fiducial 901 may contain a series of 6 possible bands 903 that indicate a binary code between 000000₂ and 111111₂, i.e. 0 to 63₁₀, to identify up to 32 different fiducials. In this case, the missing mark in position 2 (item 904 in this figure) indicates that this fiducial is identified as item 905 which is decoded from the bands as 101111₂ or 32+0+8+4+2+1=47₁₀. Other radio-opaque markings are possible including dots, channels, tubes, lines of distinct shapes or even the numerals themselves engraved using a radio-opaque material. Radial stripes that encode a binary or other form of number may also be used. These may be filled with radiopaque material (such as barium, tantalum, gold, stainless steel or other radio-opaque material) dots at a particular location. Other types of markings and materials are possible for other imaging modalities.

In an embodiment, a fiducial may be rendered visible on an imaging modality through activation of the fiducial. For example, the fiducial may be made to vibrate at a specific frequency so that it may be highly visible on ultrasound. By activating a specifically selected fiducial, all other fiducials may be weakly reflective on ultrasound, but the selected fiducial may be highly visible, thus enabling the physician to keep track of which fiducial will affect which region of the tissue. In another embodiment, HEF's may be sequentially activated and a temperature sensitive MRI scan may be used to identify which fiducial is which by scanning for the heat signature of the active fiducial. In an embodiment, the fiducial can be made to resonate at a frequency that appears bright on an MR scan. As each fiducial is activated, it is identified on the images until all relevant fiducials are matched one to one between location in the tissues and activation code. In an embodiment, the fiducials may be displayed on a labelled map of all the fiducials overlayed on images of the patient tissues. In an embodiment, this information may be used to plan an intervention, deciding which fiducials may be activated, for what duration etc. and which fiducials are not activated. In an embodiment, the mode of rendering the fiducial visible on the imaging modality may not be the same as the primary mode of operation to cause cell death.

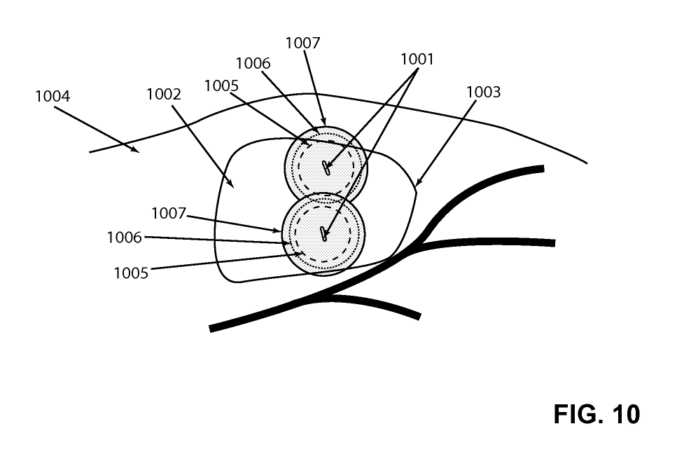

In an embodiment, fiducials may overlap as shown on FIG. 10. In this figure, fiducials 1001 are embedded in tumor 1002 with boundaries denoted by 1003. The tumor 1002 is within normal tissue 1004. Depending on the proximity of the fiducials, the tumor may be destroyed by selecting various overlapping fiducials. For example in the figure shown, three zones are shown surrounding each fiducial 1001. Zone 1005 may denote the boundary of the “kill zone” within which, all tumor cells may be killed for example by heating the fiducial for one minute. Zone 1006 may denote the boundary of the “kill zone” within which, all tumor cells may be killed for example by heating the fiducial for two minutes. Zone 1007 may denote the boundary of the “kill zone” within which, all tumor cells may be killed for example by heating the fiducial for three minutes. The union of two or more kill zones for the heating duration may therefore describe the total region of killed cells. In this example, if both fiducials are heated for three minutes, all cells within the total region contained by both circles 1007 would be destroyed.

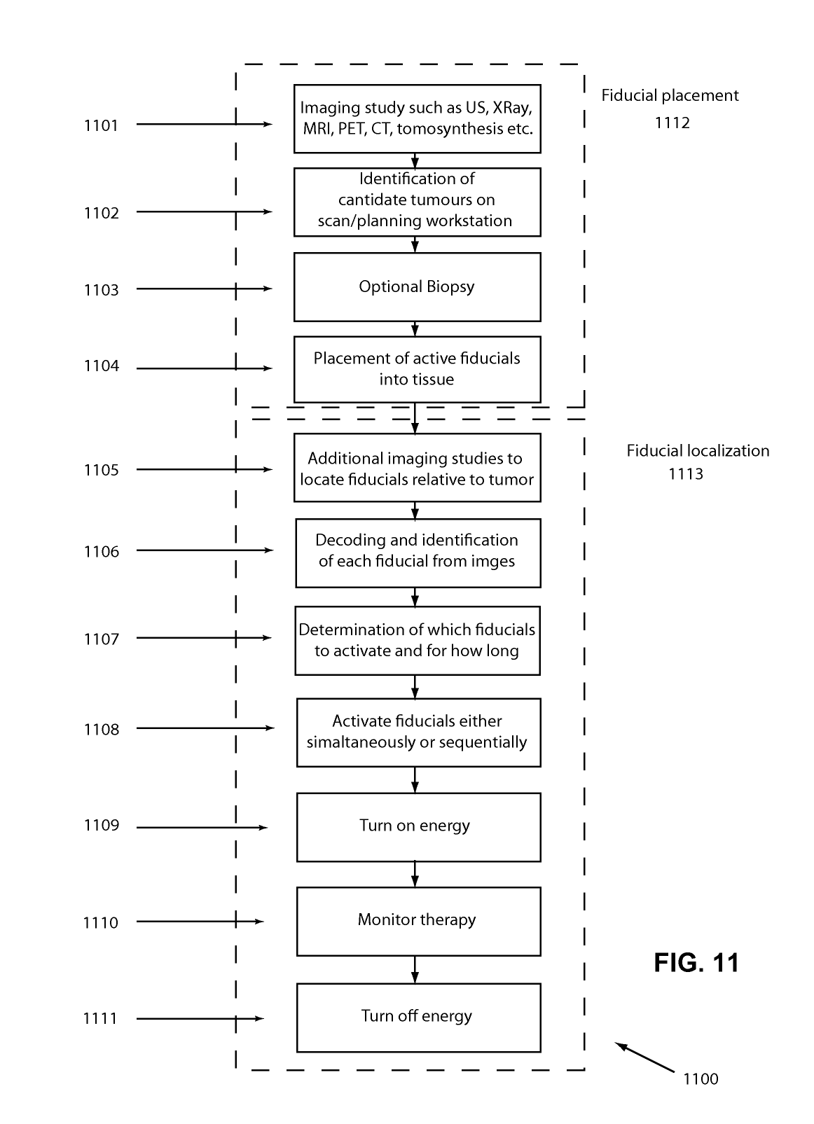

FIG. 11 illustrates a method for placing fiducials and activating them to perform therapy. The flowchart 1100 is divided into two sections, a set of fiducial placement procedures 1112 and a set of fluid localization procedures 1113.

In 1101, an imaging modality such as X-ray, MRI, CT, ultrasound, tomosynthesis, PET, can be used to obtain 2D or volumetric images of a patient's anatomy. In 1002, a lesion such as a tumor may be identified on the images. The extents of the tumor may be recorded on a planning computer, paper plan or some other medium. Critical structures may also be determined at that time.

In 1103, a biopsy optionally may be performed on the tissue to confirm its nature. The biopsy, if performed may be done under image guidance such as CT, MRI, Fluoroscopy, ultrasound etc.

Fiducials may be placed into the tissue, again with the assistance of imaging as shown in 1104. Depending on the approach being taken, fiducials may be placed around the tumor, within the tumor, both inside and outside the tumor. Fiducials may also be placed at the location of critical structures. Once the fiducials are placed, the patient may be removed from the imaging system or even sent home until the fluid localization, or interventional procedure some time later.

At the time of the fluid localization or intervention 1113, additional image studies may be performed as shown in 1105. These studies may confirm the location of the fiducials relative to the pathology and critical structures. In 1106, the fiducials can be studied on the images to identify the markings on each and correlate them with the known markings for each fiducials. The location and identity of each fiducial may then be understood in terms of the location of the tumor.

In 1107, a plan may be made on which fiducials should be activated and in what manner in order to optimally treat the tumor. For example the temperature and duration to raise a particular fiducial to may be determined. In 1108, one or more fiducials may be activated. In an embodiment, all fiducials may be activated simultaneously and in another embodiment, a subset of fiducials may be activated sequentially. In 1109, the energy required to treat the tumor may be turned on, treating the tissue. In 1110, a method may be used to monitor the therapy. In an embodiment, the temperature, emitted light, radiation or other aspect may be monitored either directly or indirectly.

In 1111, the energy may be terminated once the desired effect has been reached. If necessary additional fiducials may be selected and turned on and procedures 1109-1111 may be repeated until all the fiducials that need to be activated to perform the plan determined in 1107 have been activated.

In an embodiment, the fiducials may include a localization component that allows the (x,y,z) location and/or orientation of the fiducials (relative to the probe) to be determined by a position sensor (indicated as 102 on FIG. 1). This may be in addition to or completely separate from some of the functionality listed above. In this embodiment, a position detector detects the location and orientation of the fiducial using time of flight, intensity or other effects known in the art. A plurality of receive coils may be used to determine this position and orientation.

In an embodiment a heat emitting fiducial may be combined with a infrared detector to locate the fiducial. In an embodiment the fiducial may be constructed to measure chemical properties, electrical properties, temperature, pressure, pH, drug levels, glucose levels etc. In an embodiment, a hybrid fiducial may be produced which has one or more characteristics of the aforementioned fiducials. In an embodiment the seeds contain one or more piezoelectric elements that assist with measuring pressure or when rapidly cycled may cause the see to heat up. In an embodiment in a prostate application the devices may be used to either treat cancer or benign prostate hyperplasia (BPH).

What is claimed:

1. A method for performing a guided interventional procedure, the method comprising:
a) fiducial placement comprising:
performing an imaging study on a tissue;
identifying a lesion in the tissue;
placing one or more fiducials in the tissue;
b) fiducial localization comprising:
placing a probe in proximity to the one or more fiducials in the tissue, wherein the probe is configured to detect the one or more fiducials;
detecting the one or more fiducials;
indicating the position of the probe relative to the one or more fiducials; and
performing a procedure on the tissue;
wherein the one or more fiducials are devices configured to emit a signal in response to a signal transmitted by a probe.

2. The method of claim 1, wherein the one or more fiducials are detected by a technology selected from the group consisting of Very Low Frequency (VLF) metal detection technology, a Pulse Induction (PI) metal detection technology, a Beat-frequency Oscillation (BFO) metal detection technology, Radio frequency identification (RFID) technology, radar technology, micropower impulse radar (MIR) technology, sonic technology, optical technology, or combinations thereof.

3. The method of claim 1, wherein more than one fiducial is placed in the tissue, and wherein at least one of the fiducials comprises a property selected from the group consisting of size, shape, surface area, material, magnetic, thermal, optical, sonic, chemical, or combinations thereof, that is differentiable from at least one other fiducial.

4. The method of claim 1, wherein the imaging study is selected from the group consisting of ultrasound (US), contrast enhanced US (CEUS), x-ray, biplane x-ray, cone beam x-ray, magnetic resonance imaging (MRI), computed tomography (CT), tomosynthesis, positron emission tomography (PET), and magnetic particle imaging.

5. The method of claim 1, wherein the tissue comprises one or more of breast tissue, liver tissue, pancreas tissue, prostate tissue, brain tissue, or bone tissue.

6. The method of claim 1, wherein the guided interventional procedure is selected from the group consisting of resecting the tissue, injecting drugs or other therapeutic agents, ablation using heat, laser, or cryotherapy, placement of additional fiducials, or positioning of a therapeutic energy or radiation beam on the tissue.

7. The method of claim 1, further comprising activating at least one of the one or more fiducials in the tissue.

8. The method of claim 7, wherein activating the at least one of the one or more fiducials causes the fiducial to emit heat, light, sound, vibration, or combinations thereof.

9. The method of claim 1, wherein the position of the probe relative to the one or more fiducials is indicated on a display.

10. A fiducial device configured to emit a signal indicative of its location relative to a probe in response to a signal transmitted by a probe where the fiducial is comprised of:
a) an implantable device selected from the group consisting of a reflective device capable of reflecting an incident signal, a device designed to transmit a signal in response to an incident signal, a device designed to modify a property of a signal generating system, and combinations thereof; and
b) an element configured to constrain the fiducial within a tissue.

11. The fiducial device of claim 10, wherein the reflective device comprises a radar reflector, an optical reflector, a retro-reflector, or a phase conjugate mirror.

12. The fiducial device of claim 10, wherein the device designed to transmit a signal includes a conductor, an inductor, a capacitor, an oscillator, an active (battery-powered) RFID device, or a passive RFID device.

13. The fiducial device of claim 10, wherein the element configured to constrain the fiducial comprises ridges.

14. The fiducial device of claim 10, wherein the element configured to constrain the fiducial comprises at least one barb.

15. The fiducial device of claim 10, wherein the fiducial is coated with a glass bead coating, a prismatic coating, or an echogenic coating.

16. The fiducial device of claim 10, further comprising an antenna configured to receive or emit signals from or to an external device.

17. The fiducial device of claim 10, further comprising a light source, a radio source, a microwave source, or an audio source.

18. A system for performing a guided interventional procedure, comprising:
a computer;
a tracking device configured to communicate with the computer;
an imaging device configured to communicate with the computer; and
one or more fiducials;
wherein the one or more fiducials is configured to emit a signal in response to a signal transmitted by a probe indicative of its location relative to the probe.

19. The system of claim 18, wherein the imaging device is selected from the group consisting of an x-ray equipment, a computerized tomography equipment, a positron emission tomography equipment, a magnetic resonance imaging equipment, a fluoroscopy equipment, an ultrasound equipment, an isocentric fluoroscopic device, a rotational fluoroscopic reconstruction system, a multislice computerized tomography device, an intravascular ultrasound imager, an optical coherence tomography (OCT) device, an optical imaging device, a single photon emission computed tomography device, and a magnetic particle imaging device.

20. The system of claim 18, wherein the tracking device is selected from the group consisting of an electromagnetic tracking device, a micropower impulse radar (MIR) or other radar device, an optical range finder, a metal detector, an ultrasonic tracking device, a global positioning system (GPS) enabled tracking device, and a fiber-optic tracking device.

21. The system of claim 18, wherein the fiducial is placed on or integrated in a needle, a biopsy needle, a catheter, a clamp, a heart valve, a total joint arthroplasty, an intramedullary nail, a medical probe, or a mesh.

22. The system of claim 18, wherein the system comprises more than one fiducial, and wherein at least one of the fiducial comprises a property selected from the group consisting of size, shape, surface area, material, magnetic, thermal, optical, sonic, chemical, or combinations thereof, that is differentiable from at least one other fiducial.
